SEQUENCE LISTING (25 sequences)

SEQ ID NO: 1 - protein, 23 aa, Artificial
  cyclic analogue of histatin
DSHAKRHHGYKRFHEKHHSHRGY

SEQ ID NO: 2 - protein, 23 aa, Artificial
  cyclic analogue of histatin
DSHAKRHHCYKRFHEKHHSHRCY

SEQ ID NO: 3 - protein, 19 aa, Artificial
  cyclic analogue of histatin
RHHCYKRKFHEKHHSHRCY

SEQ ID NO: 4 - protein, 19 aa, Artificial
  cyclic analogue of histatin
RHHCYKRKFHEKHHSHRGC

SEQ ID NO: 5 - protein, 19 aa, Artificial
  cyclic analogue of histatin
RHHCYKRKFHEKHHSHCGY

SEQ ID NO: 6 - protein, 19 aa, Artificial
  cyclic analogue of histatin
RHHCYKRKFHEKHHSCRGY

SEQ ID NO: 7 - protein, 19 aa, Artificial
  cyclic analogue of histatin
RHHCYKRKFHEKHHCHRGY

SEQ ID NO: 8 - protein, 19 aa, Artificial
  cyclic analogue of histatin
RHHCYKRKFHEKHCSHRGY

SEQ ID NO: 9 - protein, 19 aa, Artificial
  cyclic analogue of histatin
RHHCYKRKFHEKCHSHRGY

SEQ ID NO: 10 - protein, 19 aa, Artificial
  cyclic analogue of histatin
RHHGCKRKFHEKHHSHCGY

SEQ ID NO: 11 - protein, 19 aa, Artificial
  cyclic analogue of histatin
RHHGYKRKCHEKHHCHRGY

SEQ ID NO: 12 - protein, 19 aa, Artificial
  cyclic analogue of histatin
RHHGYKRCFHEKHHCHRGY

SEQ ID NO: 13 - protein, 19 aa, Artificial
  cyclic analogue of histatin
RHHGYKCKFHEKHHCHRGY

SEQ ID NO: 14 - protein, 19 aa, Artificial
  cyclic analogue of histatin
RHHGYCRKFHEKHHCHRGY

SEQ ID NO: 15 - protein, 19 aa, Artificial
  cyclic analogue of histatin
RHHGCKRKFHEKHHCHRGY

SEQ ID NO: 16 - protein, 19 aa, Artificial
  cyclic analogue of histatin
RHHGYKCKFHEKHHSCRGY

SEQ ID NO: 17 - protein, 19 aa, Artificial
  cyclic analogue of histatin
RHHCYKRKFHEKHHCHRGY

SEQ ID NO: 18 - protein, 18 aa, Artificial
  cyclic analogue of histatin
RHHCYKRKFHEKHHCHRG

SEQ ID NO: 19 - protein, 17 aa, Artificial
  cyclic analogue of histatin
RHHCYKRKFHEKHHCHR

SEQ ID NO: 20 - protein, 16 aa, Artificial
  cyclic analogue of histatin
RHHCYKRKFHEKHHCH

SEQ ID NO: 21 - protein, 15 aa, Artificial
  cyclic analogue of histatin
RHHCYKRKFHEKHHC

SEQ ID NO: 22 - protein, 20 aa, Artificial
  cyclic analogue of histatin
DRHHCYKRKFHEKHHCHRGY

SEQ ID NO: 23 - protein, 19 aa, Artificial
  cyclic analogue of histatin
RHHCYKRKFHEKHHCHRGY

SEQ ID NO: 24 - protein, 19 aa, Artificial
  cyclic analogue of histatin
RHHCWKRKFHEKHHCHRGY

SEQ ID NO: 25 - protein, 18 aa, Artificial
  cyclic analogue of histatin
RHHGYKRFHEKHHSHRGY

(19) United States
(12) Patent Application Publication
(10) Pub. No.: US 2014/0065119 A1
(43) Pub. Date: Mar. 6, 2014

(54) METHODS AND COMPOSITIONS COMPRISING CYCLIC ANALOGUES OF HISTATIN 5 FOR TREATING WOUNDS

(71) Applicants: Gilles Andre Lajoie, Waterloo (CA); Greg John Adams Vilk, London (CA); Ian Welch, London (CA)

(72) Inventors: Gilles Andre Lajoie, Waterloo (CA); Greg John Adams Vilk, London (CA); Ian Welch, London (CA)

(73) Assignee: THE UNIVERSITY OF WESTERN ONTARIO, London, ON (CA)

(21) Appl. No.: 13/884,715

(22) PCT Filed: Nov. 10, 2011
(86) PCT No.: PCT/CA2011/001265

§ 371 Date: Jul. 25, 2013

Related U.S. Application Data
(60) Provisional application No. 61/412,056, filed on Nov. 10, 2010.

(51) Int. Cl. A61K 38/12; A61K 45/06; A61K 38/16

(52) U.S. Cl. CPC A61K 38/12; A61K 38/16; A61K 45/06
USPC 424/93.72; 514/21.1; 514/9.6; 514/8.2; 424/93.7

(57) ABSTRACT

Compositions and methods for treating wounds are provided. The compositions include cyclic analogues of histatin (5).

### FIELD OF THE INVENTION

The present invention relates generally to wound treatment, and more particularly, to the use of cyclic analogues of histatin to treat wounds.

### BACKGROUND OF THE INVENTION

The healing of a wound requires a well orchestrated integration of the complex biological and molecular events of cell migration, cell proliferation, and extracellular matrix (ECM) deposition. Cellular responses to inflammatory mediators, to growth factors and cytokines, and to mechanical forces must be appropriate and precise. These fundamental processes are similar to those guiding embryogenesis, tissue and organ regeneration, and even neoplasia. However, definite differences exist between adult wounds and these other systems. In cutaneous injuries that heal readily and do not have an underlying pathophysiological defect (acute wounds), the main evolutionary force may have been to achieve repair quickly and with the least amount of energy. Hence, such wounds heal with a scar and no regeneration. In wounds with preexisting pathophysiological abnormalities (chronic wounds, such as diabetic ulcers), evolutionary adaptations have probably not occurred, impairing healing as a result.

Fibroblast/keratinocyte co-culture is one example of a wound treatment. The 12 week healing rate in both intervention and control groups was higher in the study of fibroblast/keratinocyte co-culture (56% and 38%, respectively) than those reported for dermal fibroblast culture, although a marginal effect is achieved with fibroblast/keratinocyte treatment. With regard to dermal fibroblast culture, one study reported that weekly applications of dermal fibroblast culture improved healing of plantar non-ischaemic ulcers by 12 weeks when the highest dose was compared with saline-moistened gauze, while another study found no difference between intervention and placebo. Despite the indication of wound healing efficacy, the percentage of healing in the intervention group was only 30% as compared with18% in controls. Thus, this method lacks sufficient efficacy for the cost involved.

Skin grafting may also be used in wound treatment. In this regard, a comparison of meshed autologous and conventional split-skin grafts appeared to indicate little difference between the two methodologies.

Growth factor wound treatments are also known in the art, including basic fibroblast growth factor (bFGF) and epidermal growth factor (EGF) treatments. EGF has been shown to accelerate epidermal growth in experimental wounds. Two studies reported a significantly higher rate of healing of ulcers (mainly of the foot) when compared to placebo; however, another less robust study on patients with leg ulcers reported contradictory results. Growth factor treatment to date, thus, appear to be largely ineffective and expensive.

In view of the foregoing, it would be desirable to develop an effective method of treating wounds.

### SUMMARY OF THE INVENTION

Cyclic analogues of histatin 5 have now surprisingly been found to have wound-healing properties.

Thus, in one aspect of the present invention, there is provided a method of treating a wound in a mammal comprising administering to the wound a therapeutically effective amount of a cyclic analogue of histatin 5 and functionally equivalent derivatives thereof.

In another aspect of the invention, a composition useful for wound treatment is provided comprising a cyclic analogue of histatin 5 or and a functionally equivalent derivative thereof in combination with a pharmaceutically acceptable carrier.

In a further aspect, an article of manufacture is provided comprising packaging and a composition comprising a cyclic analogue of histatin 5 or and functionally equivalent derivative thereof. The packaging is labelled to indicate that the composition is suitable for wound treatment.

In a further aspect of the invention, a cyclic histatin 5 or a functionally equivalent derivative thereof is provided for use in the manufacture of a medicament for wound treatment.

These and other aspects, features and advantages of the invention will become apparent from the following detailed description, claims and drawings.

### BRIEF DESCRIPTION OF THE DRAWINGS

FIG. 1 illustrates the amino acid sequences of histatin 5 and cyclic analogues thereof;

FIG. 2A is a table denoting progression of healing using different cyclic histatin treatments on 6 separate parameters (Lesion size, Granulation tissue, Inflammation, Mineralization, Scab formation, and Depth of Lesion) rated for severity using a scoring system of 0 (not present), 1(mild), 2 (moderate) and 3 (severe);

FIG. 2B graphically illustrates the results of wound healing treatment with cyclic histatin treatment;

FIG. 3 graphically illustrates the lack of toxicity of a cyclic histatin analogue on human primary cultured cells as compared with the toxicity of ketoconazole; and

FIG. 4 graphically compares the wound healing activity of a cyclic histatin analogue with polysporin.

### DETAILED DESCRIPTION

A method of treating a wound in a mammal is provided. The method comprises administering to the wound a therapeutically effective amount of a cyclic histatin 5 analogue or a functionally equivalent derivatives thereof.

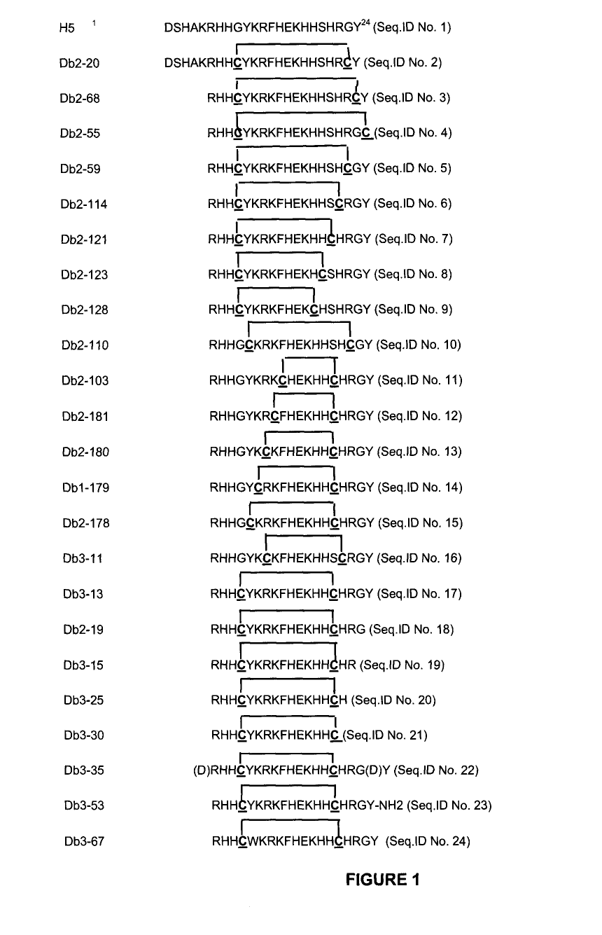

Histatin 5 is an anti-microbial peptide having the amino acid sequence, ¹DSHAKRHHGYKRFHEKHHSHRGY²⁴ (SEQ ID No: 1). Cyclic histatin 5 analogue is a cyclized version of histatin 5, e.g. which incorporates an internal bond between residues within histatin 5 that are spaced about 5 to 14 amino acid residues apart to result in cyclization of the histatin 5. Amino acid residues within histatin 5 may be substituted with one or more residues suitable to result in a cyclization thereof, for example, substitution with cyclizable amino acid residues such as lysine, glutamic acid or cysteine residues to provide a histatin 5 analogue that is readily cyclized using these residues. Thus, suitable analogues of histatin 5 exhibit substantial sequence homology with histatin 5, for example, at least about 90-95% sequence homology. An example of such an analogue is DSHAKRHHCYKRFHEKHHSHRCY (SEQ ID No: 2). It will be appreciated that other suitable cyclic analogues may include cyclizable residues at different positions within the histatin 5 sequence. Other suitable positions are illustrated in FIG. 1. Functionally equivalent derivatives are also encompassed which include truncated versions of histatin 5 with suitable substitutions to include cyclizable amino acid residues as well as derivatives that include additional terminal amino acids at either end thereof. The term “functionally equivalent” refers to derivatives that retain the activity of cyclized histatin 5 to heal wounds. The term “derivative” also encompasses modifications at a reactive sites such as at a free carboxyl or amine group or other side chain group. Such modifications may be implemented in order to confer on the histatin analogue or derivative desirable properties such as increased stability, or improved cellular uptake. In one embodiment, the cyclic histatin analogue comprises the sequence RHHGYKRFHEKHHSHRGY (SEQ ID No. 25) in which one or two of the amino acid residues is substituted with an amino acid selected from the group consisting of cysteine, glutamic acid, lysine and a thiol-containing amino acid to permit cyclization.

The preparation of cyclic analogues of histatin 5 is described in U.S. Patent No.6,555,650, the contents of which are incorporated herein by reference, and generally involves standard methods of peptide synthesis followed by a cyclization reaction to provide cyclic analogues. Histatin 5 or variants thereof are modified to incorporate cyclizable amino acid residues, e.g. residues suitable to form a cyclized histatin analogue, for example lysine, glutamic acid and/or cysteine, which are suitably spaced, e.g. about 5 to 14 amino acid residues apart to form a cyclized loop that is about 7 to 16 amino acid residues in length. The cyclized analogue may then be prepared. The cyclization reaction may involve the reaction of a free amino group on a lysine residue with a free carboxyl group on a glutamic acid residue to form a lactam (amide) link that results ring formation. However, the glutamic acid residue naturally present in histatin 5 at position 16, should preferably be left intact and not utilized in the cyclization as it appears to play a role in the activity of the final compounds. Accordingly, it is preferred to introduce a glutamic acid as a substitute amino acid group for purposes of reaction with lysine to form a cyclized analogue via a lactam group. Lysine, for participation in the cyclization reaction, may also be introduced as a substitute group, or alternatively, a lysine that naturally exists within a histatin may be utilized.

Cyclization to form a cyclized histatin analogue may also be achieved by reaction of cysteine residues within histatin, either naturally existing within a histatin or introduced by substitution. Cysteine residues in the histatin peptide chain readily react to form disulfide bridges that impart the necessary cyclic structure. While the bonds are chemically reversible, they are sufficiently permanent to meet the criteria for compounds of the present invention. As alternatives to cysteine, other thiol-containing amino acids e.g. homocysteine or penicillamine, may also be utilized as substitute amino acids. Bicyclic analogues with two disulfide bridges or one disulfide and one lactam bond may also be made and used in accordance with the invention, as well as head-to-tail cyclized analogues. Cyclization may also be accomplished by other chemical means, e.g. by thioether linkage.

Peptides containing cysteine substitutions and cyclized through disulfide groups can be made by air oxidation of the free linear peptide in a solution of ammonium bicarbonate (e.g. 0.1 M), with selective protection of the cysteine. Lactam-cyclized peptides can be prepared by the selective removal of the Aloc/Allyl Lys and Glu side chain protecting groups under mild conditions with a Pd(0) catalyst while the peptide is still attached to the resin and the other side chains remain protected. An amide bond can then be formed between the side chains using (benzotriazolyloxy) tris(dimethylamino) phosphonium hexafluorophosphate (pyBOPO and HOBt as coupling reagents, or with other common coupling reagents.

The cyclic histatin-5 analogues anad derivatives are useful to treat wounds in a mammal. The term “wound” is used herein to refer to any injury of the skin, including both open and closed wounds, such as an incision, laceration, abrasion, puncture wound, penetration wound, wound caused by blunt force trauma, burns, as well as wounds to mucosal surfaces such as oral, ocular and vaginal surfaces and chronic wounds including skin ulcers such as pressure, arterial, venous and diabetic ulcers. The wound may or may not result from an infection by a microorganism or may or may not be characterized by the presence of a microorganism

The term “treat” as it is used herein with respect to a wound refers to the amelioration or healing of a wound. Wound healing may be measured based on parameters such as lesion size, granulation tissue, inflammation, mineralization, scab formation and depth of lesion, and thus may be evident by the extent of improvement in one or more of these parameters, including the extent of wound closure. Thus, an improvement in one or more of these parameters of at least about 10%, or wound closure of at least about 10%, is indicative of wound healing. A cumulative score of these parameters visually observed (wherein 0 is healthy, 1 is mild, 2 is moderate and 3 is severe) may also be determined to determine wound healing. Thus, a decrease in the cumulative score of these parameters in a wound is indicative of wound healing, e.g. a decrease in the cumulative score of about 10%, preferably 20% and more preferably 30% or greater, is indicative of wound healing.

The term “mammal” is used herein to encompass both human and non-human mammals.

Therapeutically effective dosages of cyclic histatin 5 analogues or derivatives are administered to a mammal to treat a wound. The term “therapeutically effective” as it is used herein with respect to dosages refers to a dosage that is effective to treat a given wound without causing unacceptable adverse side effects. The term “administered” refers to any appropriate means of providing the cyclic histatin dosage to a recipient, and will depend on the dosage form being used as will be described. For example, the dosage may be administered orally, by injection, mucosally and topically as will be described in more detail.

Therapeutically effective dosages according to the method, thus, are in the range of 0.01 mg to about 100 mg per kg body weight, for example, in a range of about 0.05 mg to about 50 mg per kg. However, as one of skill in the art will appreciate, the effective therapeutic dosage of the histatin 5 cyclic analogues or derivatives will vary depending on the symptoms, age and body weight of the patient being treated, the nature and severity of the wound to be treated, the histatin analogue used and the route of administration. The present histatin 5 analogues or derivatives may be administered in a single dose or in divided doses.

The cyclic histatin 5 analogues or derivatives may be administered in the treatment of a wound alone or in a composition combined with a pharmaceutically acceptable adjuvant or carrier. The expression “pharmaceutically acceptable” means acceptable for use in the pharmaceutical arts, i.e. not being unacceptably toxic, or otherwise unsuitable for administration to a mammal. Examples of pharmaceutically acceptable adjuvants include, but are not limited to, diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule, lozenge, solution or suspension in an aqueous or non-aqueous liquid, an oil-in-water or water-in-oil liquid emulsion, an elixir or syrup are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, disintegrating agents, anti-oxidants, preservatives, colouring agents and flavouring agents may also be present. In another embodiment, the cyclic analogue may be formulated for application topically as a cream, lotion or ointment. For such topical application, the cyclic analogue is combined with an appropriate base such as a triglyceride base. Such creams, lotions and ointments may also contain a surface active agent and other cosmetic additives such as skin softeners and the like as well as fragrance. Aerosol formulations, for example, for nasal delivery, may also be prepared in which suitable propellant adjuvants are used. Compositions of the present invention may also be administered as a bolus, electuary, or paste. Compositions for mucosal administration are also encompassed, including oral, nasal, rectal or vaginal administration for the treatment of wounds in these areas. Such compositions generally include one or more suitable non-irritating excipients or carriers comprising, for example, cocoa butter, polyethylene glycol, a suppository wax, a salicylate or other suitable carriers. Other adjuvants may also be added to the composition regardless of how it is to be administered which, for example, may aid to extend the shelf-life thereof.

In particular embodiments, cyclic histatin 5 analogues or derivatives may be topically applied to a wound admixed with an absorbent hydrogel material such as lyophilized collagen. The histatin analogue may also be affixed to a polymer or other matrix, e.g. such as a bandage or a polymer mesh that is suitable for application directly onto the wound. Dermal fibroblasts/keratinocytes bioengineered to express a wound healing histatin 5 analogue or derivative may also be applied to a wound. A suitable matrix or polymer mesh, e.g. artificial or non-artificial skin grafts, may alternatively be impregnated with a selected histatin 5 analogue or derivative for application to a wound to permit slow-release of the histatin 5 analogue or derivative for continuous treatment of the wound over a period of time.

In another aspect, the present cyclic analogues or derivatives of histatin 5 may be administered to a mammal in need of wound treatment in combination with one or more additional therapeutic agents, including for example, a wound healing agent such as a growth factor, e.g. epidermal growth factor, bFCF, PDGF; platelets, dermal fibroblasts and keratinocytes. In this regard, the cyclic histatin 5 analogue or derivative may be administered to a mammal in the treatment of a wound either individually in separate formulations, or together in a combined formulation.

The present wound healing histatin 5 analogues or derivatives may be further utilized in a combination therapy in which laser therapy, for example, is applied to the wound site with repeated applications of a cyclic histatin peptide to effect wound healing.

In a further aspect of the invention, an article of manufacture is provided comprising packaging and a composition comprising a cyclic analogue or derivative of histatin 5 as described. The packaging is labelled to indicate that the composition is suitable for wound treatment.

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the claims that follow.

Embodiments of the invention are described by reference to the following specific examples which are not to be construed as limiting.

### EXAMPLE 1

### Activity of DB2121 to Treat Wounds

MATERIALS AND METHODS: DB2121, a cyclic derivative of histatin 5 was synthesized in-house using a peptide synthesizer and established methods. DB2121 was confirmed to be a cyclic peptide (as shown in FIG. 1) of mass 2558.23 g/mol with the amino acid sequence N-RHHCYKRKFHEKHHCHRGY-C. The compound was then purified using HPLC to a purity of at least 95% and lyophilized. Twenty milligrams of DB2121 was then resuspended in 10 mM Tris-Cl, pH 7.2, air oxidized overnight at 4° C. then used as a stock solution for subsequent experiments. Exact mass was verified by direct injection into a Micromass ESI-Qtof Quattro Micro mass spectrometer operated by MassLynx 4.0 software. Ketoconazole was purchased from Sigma Chemical Co. The Balb/c mice were supplied by Charles River and were housed in the Universities' Animal Care Facility. The mice were acclimated first for a minimum of 48 hours to confirm health prior to experimentation. Pentobarbital, Euthanyl, Buprenorphine and chlorhexidine were purchased from Sigma Chemical Co. All procedures performed on the animals were approved by the Animal Care and Use Committee at the University of Western Ontario.

Balb/c mice were weighed on the day of the surgery and anesthetized with isoflurane gas by being placed in a chamber and gas administered at 1 l/min oxygen and 4% isoflurane. When anesthetized they were then transferred to an in-house nose-mask system and maintained on 1 l/min oxygen and 4% isoflurane for surgery. Mice were prepped by shaving an area on the dorsal, thoracic surface. The shaved surface was then cleaned with chlorohexidine soap and alcohol. Buprenorphine at a concentration of 0.05 mg/kg was injected intraperitoneally once for pain management. Surgery then consisted of two skin defects over the dorsal, thoracic area made with a six mm circular biopsy punch. The caudally-located skin defect was filled with 25 microlitres of sterile phosphate buffered saline (PBS). The cranially-located wound was then filled with 25 microlitres of test article of varying concentrations (10mg/kg, 1 mg/kg, or 0.1 mg/kg DB2121 in sterile PBS or 10 mg/kg ketoconazole in sterile PBS). The wounds were left open and the mice were allowed to recover for 15 minutes. Then, the mice were transferred back to their individual cages and left for 7 days with ample food and water.

At the 7-day mark, the Balb/c mice were euthanized and blood extracted for future analysis. The skin wounds were then excised using a scalpel and placed immediately in room temperature formalin for fixation. After a 48-hour fixation period, the tissue samples were washed with PBS/ ethanol of various concentrations using established procedures, placed in a histology chamber and finally embedded in paraffin wax overnight. The embedded skin tissue was cut in sections to a thickness of 5 μm and placed on glass microscope slides. AU slides were finally Hematoxylin/Eosin stained and stored at room temperature.

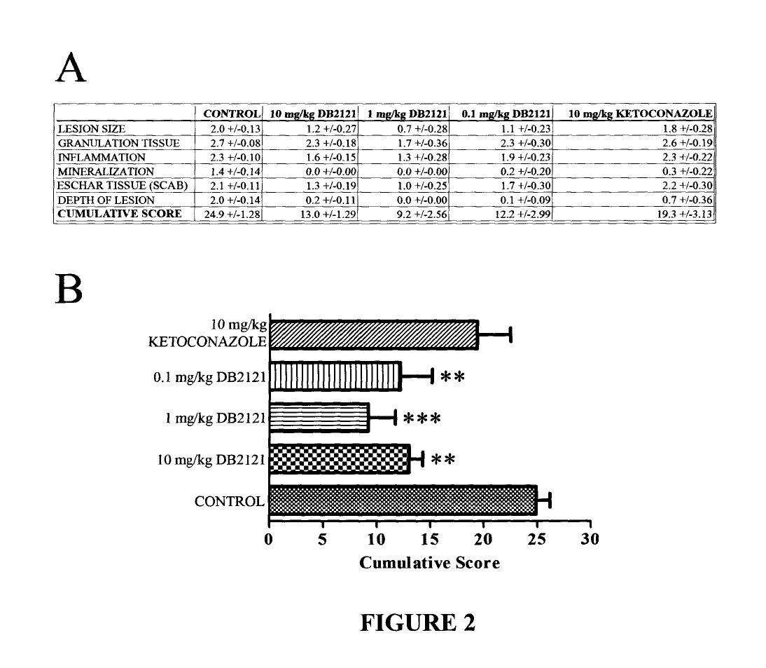

A visual scoring system was applied to gauge the progression of the wound healing process using 6 different parameters namely lesion size, granulation tissue, inflammation, mineralization, scab formation, and depth of lesion. The score for each parameter was noted in duplicate on separate sections and was based on a scale of 0 (healthy), 1 (mild), 2 (moderate) and 3 (severe). As set out in FIG. 2A, the scores for each parameter were then summed for each treatment and this final tally was termed the cumulative score; a low cumulative score meant the tissue was considered essentially healed whereas a high score meant that the wound was still in the processes of healing with varying degrees of severity. The observed mean and standard of the mean of the cumulative score were calculated for each treatment and plotted on a bar graph (FIG. 2B). One-way ANOVA analysis (each treatment vs. CONTROL treatment) using the Tukey method was employed to determine the significance of the findings. A confidence interval of at least P<0.05 was considered significant.

The results illustrated in FIG. 2A/B show that at each of the tested concentrations, DB2121 significantly improved the wound healing process (**P<0.01; ***P<0.001) with acumulative score as low as 9.2+/−2.56 at a concentration of 1 mg/kg DB2121 (cumulative score for saline control was 24.9+/−1.28). Direct observation of the histology clearly shows an improvement in the wound healing process by the introduction of DB2121 to the skin defect.

After 7 days, the control wounds appeared inflamed, still contained eschar (scab) tissue, had an incomplete epithelium and finally were peppered with dark-stained granulation; all signs of a wound that is in the early stages of the wound healing process. However, DB2121 treatment to the skin defect resulted in improved healing as illustrated by a completely-formed epithelium across the entire width of the wound, insignificant eschar tissue, reduced redness, and a lack of inflammatory cells and dark-stained granulation.

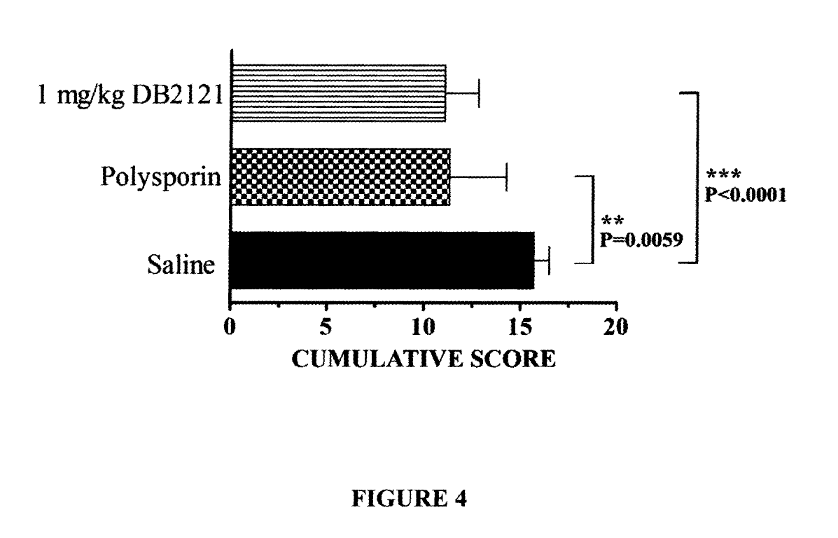

A similar experiment was conducted to compare the wound healing activity of DB2121 in comparison to an established product, polysporin, that contributes to wound healing. As shown in FIG. 4, the wound healing activity of DB2121 is comparable to that of polysporin.

### EXAMPLE 2

### Toxicology of DB2121

Toxicological studies of DB2121 in human cells were also undertaken. Primary human neonatal foreskin epithelial cells were used to examine DB2121 toxicity, using a method as described in Min et al. 2004. Nat Biotechnol 22:717-23, the contents of which are incorporated herein by reference. Briefly, when the cells were in log phase, (A) 50 μM DB2121, B) 50 μM Histatin H5, (C) 50 μM ketoconazole, or (D) DMSO was added to the culture medium. At 18 and 36 hours following addition of compound, the foreskin epithelial cells were imaged using phase contract microscopy at 20× magnification. Immediately following the image analysis, proteins were extracted, separated on SDS-PAGE gels, Western blotted, and probed for phosphorylated and non-phosphorylated ERK 1, 2 to monitor cell spreading/migration and/or proliferation of the foreskin keratinocytes. The experiments was repeated three times.

Following the foregoing treatment, the human primary cells appeared morphologically normal except when treated with ketoconazole. The toxicity of ketoconazole to mammalian cells has been noted previously.

It was then investigated whether or not these phenotypes were reflected at the molecular level. The primary cells were lysed and monitored for a marker of proliferation namely, phosphorylated ERK 1, 2. Significant inhibition of ERK 1, 2 activation in the ketoconazole-treated cells was observed as compared to control. Conversely, activated ERK 1, 2 was not suppressed in DB2121-treated cells as compared to controls.

Both studies show that DB2121 is non-toxic to human primary cells at working concentrations of 50 μM.

To determine range of non-toxic concentrations of DB2121, various concentrations of DB2121, 50 μM ketoconazole or DMSO as a control were separately added to primary human foreskin epithelial cell culture. At 0, 36 and 72 hours after addition, the epithelial cells were titrated off the culture plate and the viable cell number was determined. The experiment was performed in triplicate. At 72 hours, the foreskin epithelial cells exposed to the various compounds were imaged at 20× magnification using phase contrast microscopy. For cell counting, the epithelial cells were stained with trypan exclusion stain to monitor cell viability.

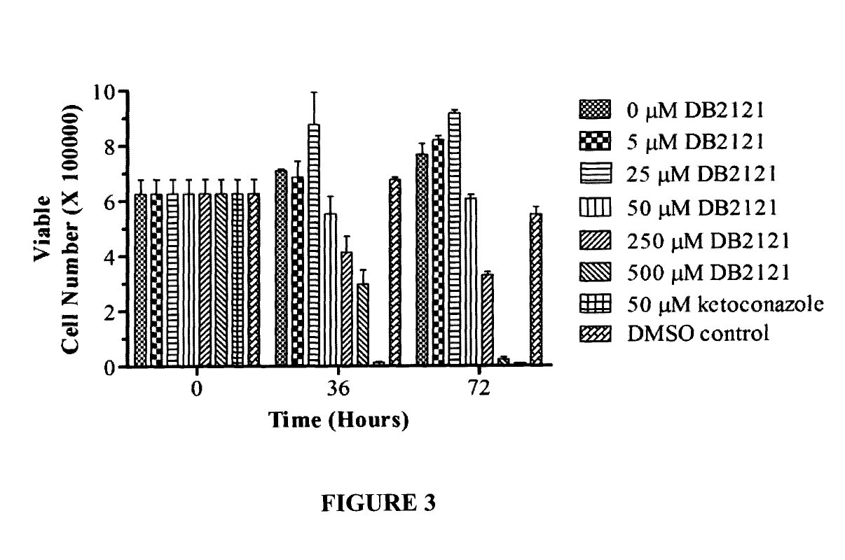

The results show minimal toxicity of DB2121 in primary cultured cells and a superior toxicity profile when compared to ketoconazole as illustrated in FIG. 3.

In addition, mice were able to withstand a concentration of 15 mg/kg of DB2121 when injected via an intra-peritoneal route. When injected into adult rats, a concentration of up to and including 1.5 mg/kg caused no harm when injected intra-venously.

### EXAMPLE 3

### Stability of DB2121

DB2121 cyclic analogue was determined to be stable in vitro in human saliva for at least 72 hours as determined by mass spectrometry. The cyclic analogue was incubated in human saliva at a concentration of 1 uM and at a temperature of 37° C. in vitro. At various time points from 0 to 72 hours, 1.0 ul aliquots were taken and injected directly into a Micromass Quattro Micro mass spectrometer. Data was collected for a total of 3 minutes. The data was then processed using Mass Lynx 4.0 Analysis software.

The expected average mass of the cyclic histatin analogue in its active form is expected to be 2557.93. Cyclic histatin analogue was shown to be present in the human saliva up to at least 72 hours incubation. The peaks within an acceptable error of approximately 1 mass unit corresponding to cyclic histatin analogue were 2556.95 at 0 hours, 2557.65 at 24 hours, and 2557.10 at 72 hours incubation.

We claim:

1. A method of treating a wound in a mammal comprising administering to the mammal a therapeutically effective amount of a cyclic analogue of histatin 5 or a functionally equivalent derivative thereof.

2. The method of claim 1, wherein the cyclic analogue comprises the sequence RHHGYKRFHEKHHSHRGY (SEQ ID No. 25) in which one or two of the amino acid residues is substituted with an amino acid selected from the group consisting of cysteine, glutamic acid, lysine and a thiol-containing amino acid to permit cyclization of the histatin.

3. The method of claim 2, wherein the cyclic analogue has an amino acid sequence selected from the group consisting of DSHAKRHHCYKRFHEKHHSHRCY, RHHCYKRKFHEKHHSHRCY, RHHCYKRKFHEKHHSHRGC, RHHCYKRKFHEKHHSHCGY, RHHCYKRKFHEKHHSCRGY, RHHCYKRKFHEKHHCHRGY, RHHCYKRKFHEKHCSHRGY, RHHCYKRKFHEKCHSHRGY, RHHGCKRKFHEKHHSHCGY, RHHGYKRKCHEKHHCHRGY, RHHGYKRCFHEKHHCHRGY, RHHGYKCKFHEKHHCHRGY, RHHGYCRKFHEKHHCHRGY, RHHGCKRKFHEKHHCHRGY, RHHGYKCKFHEKHHSCRGY, RHHCYKRKFHEKHHCHRGY, RHHCYKRKFHEKHHCHRG, RHHCYKRKFHEKHHCHR, RHHCYKRKFHEKHHCH, RHHCYKRKFHEKHHC,(D)RHHCYKRKFHEKHHCHRG(D)Y, RHHCYKRKFHEKHHCHRGY-NH2 and RHHCWKRKFHEKHHCHRGY.

4. The method of claim 3, wherein one or both of the cysteine residues in the cyclic analogue is substituted with an amino acid selected from the group consisting of glutamic acid, lysine and other thiol-containing amino acids to permit cyclization.

5. The method of claim 1, wherein the cyclic analogue is prepared from histatin 5 in which at least one of the histatin amino acids is substituted with an amino acid selected from the group consisting of glutamic acid, lysine, cysteine and other thiol-containing amino acids to permit cyclization.

6. The method of claim 1, wherein the wound is an injury to the skin selected from the group consisting of an incision, laceration, abrasion, puncture wound, penetration wound, wound caused by blunt force trauma, burn, mucosal wound, pressure ulcer, arterial ulcer, venous ulcer and diabetic ulcer.

7. The method of claim 1, wherein the analogue is administered at a dose within the range of about 0.01 mg to about 100 mg per kg body weight.

8. The method of claim 1, wherein the analogue is administered with an additional therapeutic agents selected from the group consisting of epidermal growth factor, bFCF, PDGF, platelets, dermal fibroblasts and keratinocytes.

9. The method of claim 1, wherein the analogue is administered admixed with or affixed to a matrix selected from a hydrogel, a polymer, dermal fibroblasts/keratinocytes and an artificial or non-artificial skin graft.

10. The method of claim 1, wherein the analogue is administered to a wound in combination with laser therapy.

11. A composition suitable for wound treatment comprising a cyclic analogue of histatin 5 or a functionally equivalent variant thereof and at least one pharmaceutically acceptable carrier.

12. The composition as defined in claim 11, wherein the cyclic analogue comprises the sequence RHHGYKRFHEKHHSHRGY (SEQ ID No. 25) in which one or two of the amino acid residues is substituted with an amino acid selected from the group consisting of cysteine, glutamic acid, lysine and a thiol-containing amino acid to permit cyclization of the histatin.

13. The composition of claim 11, wherein the cyclic analogue has an amino acid sequence selected from the group consisting of DSHAKRHHCYKRFHEKHHSHRCY, RHHCYKRKFHEKHHSHRCY, RHHCYKRKFHEKHHSHRGC, RHHCYKRKFHEKHHSHCGY, RHHCYKRKFHEKHHSCRGY, RHHCYKRKFHEKHHCHRGY, RHHCYKRKFHEKHCSHRGY, RHHCYKRKFHEKCHSHRGY, RHHGCKRKFHEKHHSHCGY, RHHGYKRKCHEKHHCHRGY, RHHGYKRCFHEKHHCHRGY, RHHGYKCKFHEKHHCHRGY, RHHGYCRKFHEKHHCHRGY, RHHGCKRKFHEKHHCHRGY, RHHGYKCKFHEKHHSCRGY, RHHCYKRKFHEKHHCHRGY, RHHCYKRKFHEKHHCHRG, RHHCYKRKFHEKHHCHR, RHHCYKRKFHEKHHCH, RHHCYKRKFHEKHHC,(D)RHHCYKRKFHEKHHCHRG(D)Y, RHHCYKRKFHEKHHCHRGY-NH2 and RHHCWKRKFHEKHHCHRGY.

14. The composition of claim 13, wherein one or both of the cysteine residues in the cyclic analogue is substituted with an amino acid selected from the group consisting of glutamic acid, lysine and other thiol-containing amino acids to permit cyclization.

15. The composition of claim 11, wherein the cyclic analogue is prepared from histatin 5 in which at least one of the histatin amino acids is substituted with an amino acid selected from the group consisting of glutamic acid, lysine, cysteine and other thiol-containing amino acids to permit cyclization.

16. The composition of claim 11, combined with an additional therapeutic agents selected from the group consisting of epidermal growth factor, bFCF, PDGF, platelets, dermal fibroblasts and keratinocytes.

17. The composition of claim 11, wherein the analogue is admixed with or affixed to a matrix selected from a hydrogel, a polymer, dermal fibroblasts/keratinocytes and an artificial or non-artificial skin graft.

18. An article of manufacture comprising packaging and a composition comprising a cyclic analogue of histatin 5, wherein said packaging is labelled to indicate that the composition is suitable for treating a wound in a mammal.

19. The article as defined in claim 18, wherein the cyclic analogue comprises the sequence RHHGYKRFHEKHHSHRGY (SEQ ID No. 25) in which one or two of the amino acid residues is substituted with an amino acid selected from the group consisting of cysteine, glutamic acid, lysine and a thiol-containing amino acid to permit cyclization of the histatin.

20. The article of claim 19, wherein the cyclic analogue has an amino acid sequence selected from the group consisting of DSHAKRHHCYKRFHEKHHSHRCY, RHHCYKRKFHEKHHSHRCY, RHHCYKRKFHEKHHSHRGC, RHHCYKRKFHEKHHSHCGY, RHHCYKRKFHEKHHSCRGY, RHHCYKRKFHEKHHCHRGY, RHHCYKRKFHEKHCSHRGY, RHHCYKRKFHEKCHSHRGY, RHHGCKRKFHEKHHSHCGY, RHHGYKRKCHEKHHCHRGY, RHHGYKRCFHEKHHCHRGY, RHHGYKCKFHEKHHCHRGY, RHHGYCRKFHEKHHCHRGY, RHHGCKRKFHEKHHCHRGY, RHHGYKCKFHEKHHSCRGY, RHHCYKRKFHEKHHCHRGY, RHHCYKRKFHEKHHCHRG, RHHCYKRKFHEKHHCHR, RHHCYKRKFHEKHHCH, RHHCYKRKFHEKHHC,(D)RHHCYKRKFHEKHHCHRG(D)Y, RHHCYKRKFHEKHHCHRGY-NH2 and RHHCWKRKFHEKHHCHRGY.
